(19) United States
(12) Patent Application Publication
(10) Pub. No.: US 2026/0000514 A1
(43) Pub. Date: Jan. 1, 2026

(54) BONE DEFECT REPAIR APPARATUS

(71) Applicant: University of Central Oklahoma, Edmond, OK (US)

(72) Inventors: Morshed Khandaker, Edmond, OK (US); Amgad Haleem Amen, Belmont, MA (US)

(21) Appl. No.: 19/253,363

(22) Filed: Jun. 27, 2025

Related U.S. Application Data
(60) Provisional application No. 63/665,846, filed on Jun. 28, 2024.
(60) Provisional application No. 63/665,905, filed on Jun. 28, 2024.

(51) Int. Cl. A61F 2/28; A61B 17/00; A61B 17/72; A61F 2/30; B33Y 80/00

(52) U.S. Cl. CPC A61F 2/2846; A61B 17/7233; A61F 2/30771; A61B 2017/00004; A61B 2017/00526; A61F 2002/285; A61F 2002/30062; A61F 2002/3084; A61F 2002/3092; A61F 2002/30985; B33Y 80/00

(57) ABSTRACT

A repair apparatus for repairing bone defects comprises an intramedullary nail extending into spaced apart first and second bone fragments of the bone defect through the ends thereof. A nanofiber mesh scaffold is disposed about the intramedullary nail and positioned between the ends of the first and second bone fragments.

### CROSS REFERENCE TO RELATED APPLICATIONS

This application claims priority from and the benefit of U.S. Provisional Application 63/665,846 filed Jun. 28, 2024, and U.S. Provisional Application 63/665,905 filed Jun. 28, 2024, both of which are incorporated by reference.

### STATEMENT REGARDING FEDERALLY SPONSORED RESEARCH OR DEVELOPMENT

This invention was made with government support under P20GM103447 awarded by the National Institutes of Health. The government has certain rights in the invention.

### BACKGROUND

A critical size bone defect is generally defined as the smallest size intra-osseous wound in a particular bone and species of animal that will not heal spontaneously or as a defect that shows less than 10% bone regeneration during the lifetime of the animal. A defect can also be characterized as ‘critical size’ when its length deficiency exceeds two to three times its diameter. The clinical treatment of critical-size bone defects remains a challenge worldwide due to several problems associated with autologous bone grafting with metallic intramedullary nail systems, which is the current clinical gold standard for humans.

While bone tissue engineering holds promise for the regeneration of segmental bone defects, challenges still exist with regard to effective scaffold design and translational animal studies. Developing bioresorbable bone substitutes that have strength characteristics similar to cortical bone strength and have osteoinductive properties may provide an effective method to treat the clinical large segmental bone defects. Using a bioabsorbable scaffold and intramedullary nail that can be absorbable, may offer many advantages over the autologous bone substitute solutions, including reduced recovery time, load bearing during the recovery process, and no need for future surgical procedures to remove the implants due to their bioabsorbable property.

Current segmental bone loss treatment options include distraction osteogenesis, induced membrane, and vascularized fibular transplantation with an interlocked intramedullary nail, made with metal as depicted in FIG. 1B. Each of these options has its advantages, but they also have significant drawbacks and complications. To improve and hasten healing in human segmental defects, new and emerging technologies such as a tissue-engineered bone graft has been developed, which can be assembled with the intramedullary nail in the bone defect area (FIG. 1C). There is currently no standardized treatment method for critical-size segmental long bone defects for humans. Current solutions remain unsatisfactory, requiring multiple complex surgeries, with a high risk of infection, and often leaving patients with lifelong difficulties. A new solution is needed which promotes bone union while allowing weight bearing during the healing process to aid long-term results and which requires only a single minimally invasive surgery.

The prior art apparatus of FIGS. 1A-1C depict a critical defect in a tibia, which is a weight bearing bone. The solutions for critical defects for other bone structures, such as for example, a critical defect in an ulna, which is a non-weight bearing bone, may be different. The bone fragments of a tibia can be separated while the ulna is fixed so that the bone fragments cannot be moved apart. Developing bone substitutes that are bioresorbable, osteoconductive, and osteoinductive properties is currently sought in this innovation that could provide an effective method to treat critical-size bone defects.

### SUMMARY

Aspects of this disclosure are directed to the development of bone substitutes that are bioresorbable, osteoconductive, and have osteoinductive properties for treatment of critical-size bone defects. Disclosed herein are a repair apparatus and method for critical bone defects. In one embodiment, the repair apparatus comprises a biologic nanofiber mesh wrapped around a bioabsorbable intramedullary nail. In another embodiment, this disclosure is directed to the use of a fixation apparatus that comprises upper and lower discrete nail sections and a biologic nanofiber mesh scaffold. Screws may be used to fix the nail to the bone. The biologic nanofiber mesh acts as a bone scaffold. Such an apparatus and method may reduce recovery time due to the bioabsorbable property with biodegradation, allowing creep substitution with regenerative bone tissue. The bioabsorbable nanofiber mesh scaffold spans the distance between the ends of bone fragments in a critical size defect.

### DESCRIPTION OF THE FIGURES

FIG. 1A is a depiction of a tibia bone.

FIG. 1B is a depiction of a tibia bone with a critical bone defect, along with an intramedullary nail extending into the bone fragments and secured by screws.

FIG. 1C is a depiction of a tibia bone with a critical bone defect, along with an intramedullary nail extending into the bone fragments and secured by screws. A scaffold is positioned between the bone fragments to act as a bone graft.

FIG. 2 is an elevation view of an embodiment of a repair apparatus comprising a scaffold and bioabsorbable intramedullary nail.

FIG. 3 is a depiction of a critical bone defect.

FIG. 4 is an elevation view of a bioabsorbable intramedullary nail and bioabsorbable scaffold bone graft embodiment of FIG. 2 positioned to repair the critical bone defect.

FIG. 5 is an elevation view of an additional embodiment of a repair apparatus.

FIG. 6 is an elevation view of an intramedullary nail used with the embodiment of FIG. 5.

FIG. 7 is an elevation view of a critical bone defect.

FIG. 8 is a cross-section view of the repair apparatus of FIG. 5 used to repair the bone defect of FIG. 7.

### DESCRIPTION OF AN EMBODIMENT

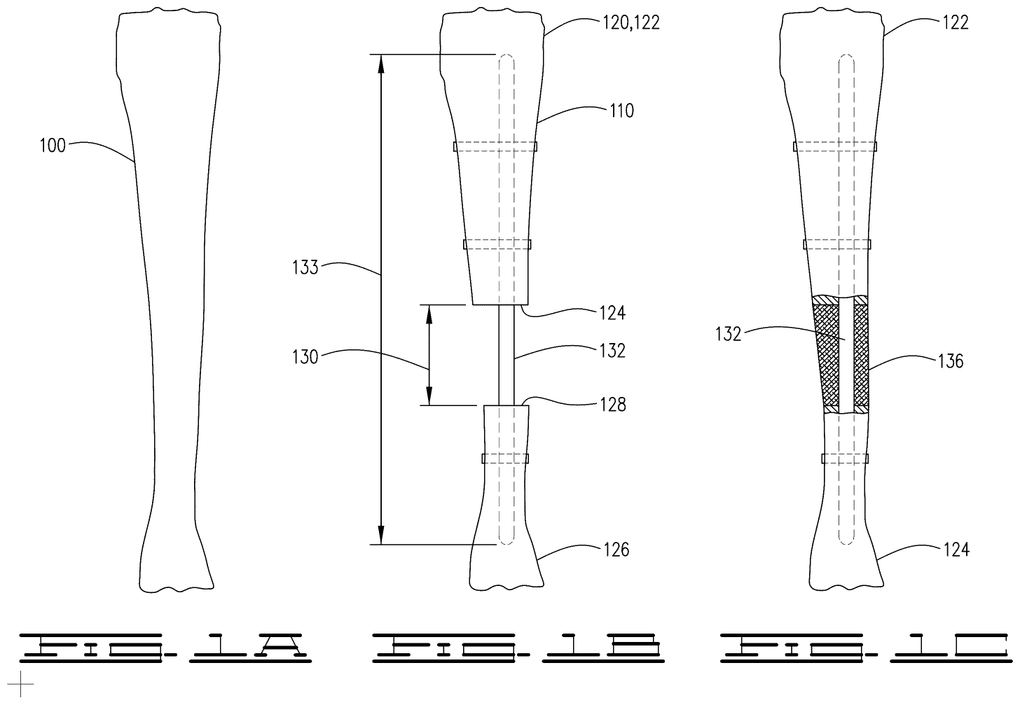

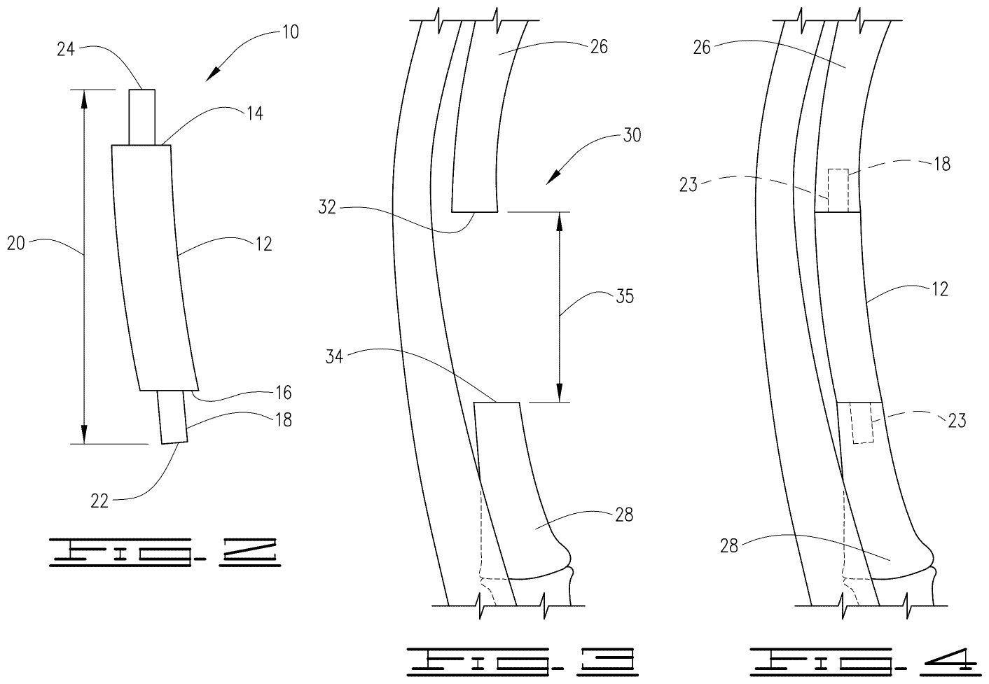

Referring now to the figures, FIG. 1A depicts a tibia 100 and FIGS. 1B and 1C depict a tibia 110 with a critical bone defect addressed by a prior art repair. The critical bone defect comprises a fracture resulting in bone fragments 120, which comprise a first, or upper bone fragment 122 with an end 124 and a second, or lower bone fragment 126 with an end 128. Bone fragments 122 and 126 have space 130 therebetween. An intramedullary metal nail 132 with a length 133 extends into the upper and lower bone fragments 122 and 126. A 3D printed scaffold bone graft 136 is positioned between the ends 124 and 128 of bone fragments 122 and 126. Upper and lower bone fragments 122 and 126 will have lengthwise holes created therein to receive the intramedullary nail 132.

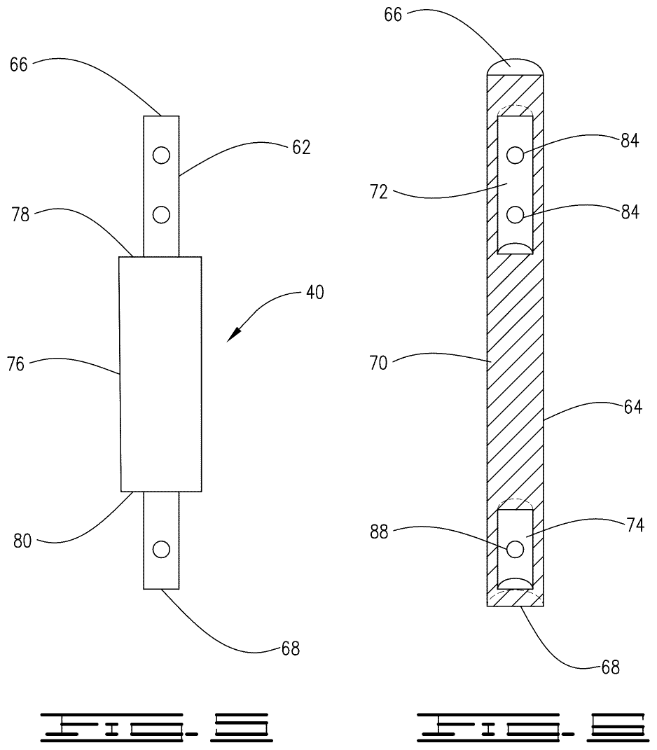

FIGS. 2-4 show an embodiment of a repair apparatus 10 that comprises a scaffold 12. Scaffold 12 may comprise a nanofiber mesh scaffold 12 having first and second ends 14 and 16. Repair apparatus 10 has fixation apparatus which may be, for example, an intramedullary nail 18, and in one embodiment a flexible, bioabsorbable intramedullary nail. The nanofiber mesh scaffold 12 is wrapped directly around the intramedullary nail 18 with no material between the nanofiber mesh scaffold 12 and the intramedullary nail 18. Intramedullary nail 18 has a length 20 extending between first and second ends 22 and 24 thereof. Intramedullary nail 18 will be inserted in and will extend into axial cavities 23 in the spaced apart first and second, or proximal and distal bone fragments 26 and 28 of bone defect 30. First bone fragment 26 has an end 32 and second bone fragment 28 has an end 34 with a space 35 therebetween. First and second ends 14 and 16 of nanofiber mesh scaffold 12 will fit between the spaced apart ends 32 and 34 of bone fragments 26 and 28.

The nanofiber mesh scaffold 12 may be produced by methods known in the art. One method involves dissolving PCL in acetone to create a solution, and feeding the solution through a single-axis, one-inch discharge metallic needle (Model #BX 25). An electrospinning machine includes a drum collector, which can be operated by speed-controlled direct current (DC) motors. A high voltage of 9 kV, generated by a high precision and high voltage power supply AC-DC conversion MAX output −20 KV 0.5 mA (Analog Technologies, Inc., San Jose, CA, USA), is applied to the syringe needle, creating an electrically charged jet in the PCL solution. The jet is directed toward the drum collector, located approximately 5 cm away from the needle at room temperature and relative humidity of 30-40%, to form a stream of synthetic polymer fibers. The parameters, such as the rotation speed of the drum, the needle-drum distance, and the fiber deposition rate, to optimize fiber mat formation. The solution feeding rate can be set as desired, and in one embodiment may be, for example, 0.025 mL/minute. The drum can be of different sizes and in one embodiment 40 mm. The foregoing is exemplary, and it is understood that the mesh can be made using other equipment and parameters. The nanofiber mesh acts as a bone graft and will be absorbed into the body of the patient and replaced by bone as the spaced apart fragments grow together.

Biomolecules can be immobilized onto nanofibers in a variety of ways including for example, encapsulation, adsorption, and covalent bonding. Encapsulated biomolecules are not attached to the nanofiber surface but are entrapped in the polymer network. The biomolecule is added to the spinning solution and becomes immobilized in the polymer matrix during the electrospinning process. In biomolecule immobilization by adsorption the biomolecule and the nanofiber substrate are placed in solution for a fixed amount of time and then rinsed with buffer solution to remove any unadsorbed biomolecules. Another method comprises covalently binding the biomolecule to the nanofiber surface. Stable complexes are formed between the functional groups of the substrate and the functional groups of the biomolecule. The binding biomolecule may be bound onto the nanofiber substrate by direct reaction onto the substrate or activation of the surface using crosslinkers. The nanofiber mesh serves as a carrier for bone marrow mesenchymal stem cells (BMSC), bone growth factors, antimicrobial agents, and heal-tracking molecules, promoting natural bone regeneration, resisting infection and monitoring healing.

Intramedullary nail 18 is a flexible, porous intramedullary nail and may be made from, for example, a bioabsorbable and biodegradable PCL material, and may be, for example, polycaprolactone-hydroxyapatite (PCL-HA). One method of manufacturing the intramedullary nail is by using a 3D printer. As shown in FIGS. 2-4, the repair apparatus 10 does not use screws or other fixation methods to attach the intramedullary nail 18 and nanofiber mesh scaffold 12 to bone fragments 26 and 28. Repair apparatus 10 was used in a rabbit ulna and is a non-weight bearing repair apparatus. The flexibility of the intramedullary nail 18 allows it to be flexed and inserted into axial openings 23 in the upper and lower bone fragments 26 and 28. A method of preparing a critical bone defect comprises fabricating an intramedullary nail from bioabsorbable and biodegradable PCL material, and depositing a nanofiber mesh on to the intramedullary nail. The length of the intramedullary nail will be greater than space between the bone fragments of the critical bone defect, and will be flexed and inserted to axial cavities in the first and second bone fragments of the critical bone defect. The intramedullary nail may also be made from other materials, for example, metals or composites.

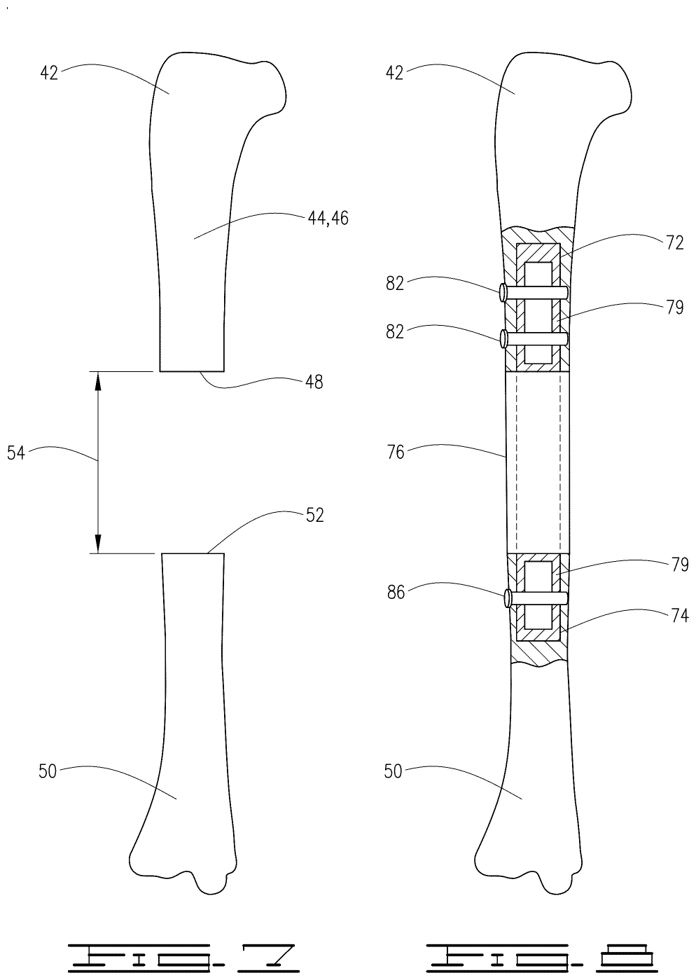

FIGS. 5-8 show an additional embodiment of a repair apparatus 40 for a critical bone defect 42. Bone defect 42 consists of bone fragments 44 that include a first, or proximate bone fragment 46 with an end 48 and a second, or distal bone fragment 50 with an end 52. Space 54 is defined between ends 48 and 52 of bone fragments 46 and 50, respectively. Repair apparatus 40 comprises a fixation apparatus 62, which may be comprised of an intramedullary nail 64 with upper and lower ends 66 and 68. Intramedullary nail 64 is shown in cross section in FIG. 6 and has a solid center nail portion 70 with upper and lower hollow shaft portions 72 and 74 connected to and extending from center nail portion 70.

Repair apparatus 40 also comprises a nanofiber mesh scaffold 76 having first and second ends 78 and 80. Nanofiber mesh scaffold 76 will fit between ends 48 and 52 of bone fragments 46 and 50 and will fill space 54. Nanofiber mesh scaffold 76 may be made in the same manner as nanofiber mesh scaffold 12. Nanofiber mesh scaffold 76 may be wrapped around the center nail portion 70. Upper and lower hollow shaft portions 72 and 74 will extend into axial cavities 79 in the spaced apart first and second, or proximal and distal bone fragments 46 and 50.

In the embodiment of FIGS. 6-8, screws 82 extend through bone fragment 46 and openings 84 in upper hollow shaft portion 72. A screw 86 will extend through bone fragment 50 and through opening 88 in lower hollow shaft portion 74. The embodiment of FIGS. 6-8 is representative of a weight bearing repair apparatus and may be used for repair of critical bone defects in, for example, a tibia or other weight bearing bone. Intramedullary nail 64 may comprise a metallic intramedullary nail or may comprise a 3D printed porous PCL intramedullary nail. If a metal intramedullary nail is used, the axial cavity 79 in lower bone fragment 50 will extend from the bottom end thereof through end 52. The described methods and apparatus provide for the repair of critical bone defects without the need for an external jig.

What is claimed is:

1. A repair apparatus for repairing bone defects consisting of spaced apart first and second bone fragments of a bone, the repair apparatus comprising:
an intramedullary nail extending into the first and second bone fragments through the ends thereof; and
a nanofiber mesh scaffold disposed about the intramedullary nail and positioned between the ends of the first and second bone fragments.

2. The repair apparatus of claim 1, the intramedullary nail comprising a flexible bioabsorbable intramedullary nail.

3. The repair apparatus of claim 1, the intramedullary nail comprising first and second spaced apart nail sections.

4. The repair apparatus of claim 3, further comprising a first screw fixing the first intramedullary nail section to the first bone segment and a second screw fixing the second intramedullary nail section to the second bone segment.

5. The repair apparatus of claim 3, wherein the nanofiber mesh scaffold is wrapped around a portion of each of the first and second spaced apart nail sections.

6. The repair apparatus of claim 1, wherein the intramedullary nail is pressed into openings in the first and second bone fragments, and no additional means are used to attach the repair apparatus.

7. A method of repairing a critical bone defect comprising:
fabricating an intramedullary nail from a porous flexible material;
depositing a nanofiber mesh on an outer surface of the intramedullary nail;
inserting a first end of the intramedullary nail into an end of first bone fragment of the critical bone defect; and
inserting a second end of the intramedullary nail into an end of second bone fragment of the critical bone defect, the first and second bone fragments defining a space therebetween.

8. The method of claim 7, wherein the intramedullary nail is comprised of PCL.

9. The method of claim 7, further comprising positioning the nanofiber mesh in the space between the first and second bone fragments.

10. The method of claim 7, further comprising fixing the intramedullary nail to the bone with fasteners.

11. The method of claim 7, wherein the intramedullary nail is comprised of a bioabsorbable material.

12. The method of claim 11, wherein the intramedullary nail is a 3D printed PCL intramedullary nail.

13. The method of claim 7, further comprising immobilizing biomolecules on the nanofiber mesh.

14. A repair apparatus for repairing bone defects consisting of spaced apart first and second bone fragments of a bone, the repair apparatus comprising:
flexible intramedullary nail inserted into the spaced apart ends of the first and second bone fragments; and
a nanofiber mesh deposited directly on the outer surface of the intramedullary nail and positioned in the space between the first and second bone fragments.

15. The repair apparatus of claim 14, the flexible intramedullary nail comprising a PCL intramedullary nail.

16. The repair apparatus of claim 14, further comprising fasteners fixing the intramedullary nail to the first and second bone fragment.

17. The repair apparatus of claim 14, wherein no fasteners are used to fix the intramedullary nail to the first and second bone fragments.
